(19) United States
(12) Patent Application Publication
(10) Pub. No.: US 2023/0320857 A1
(43) Pub. Date: Oct. 12, 2023

(54) Implant System for Treating Bone Defects or Discontinuities

(71) Applicant: Karl Leibinger Medizintechnik GmbH & Co. KG, Mühlheim/Donau (DE)

(72) Inventors: Stefanie Grom, Neuhausen ob Eck (DE); Frank Reinauer, Emmingen-Liptingen (DE); Tatjana Fecht, Mühlingen (DE); Tobias Wolfram, Dreieich (DE)

(21) Appl. No.: 18/296,469

(22) Filed: Apr. 6, 2023

(30) Foreign Application Priority Data
Apr. 8, 2022 (DE) 10 2022 203 561.5

(51) Int. Cl. A61F 2/28

(52) U.S. Cl. CPC A61F 2/28; A61F 2002/2835; A61F 2310/00592

(57) ABSTRACT

The invention provides an implant system for treating bone defects or discontinuities and a method for producing such an implant system. The implant system (100) comprises:
a first implant element (110) which is insertable or inserted into a bone defect or a discontinuity (2) of a human bone (1),
a second implant element (120) which is fixable or fixed to the human bone (1),
wherein the first implant element (110) is attachable or attached to the second implant element (120) by means of at least one biodegradable connection means (125) in order to fix the first implant element (110) relative to the human bone (1),
wherein the first implant element (110) comprises a shell section (111) and an inner section (112) at least partially enclosed by the shell section (111),
wherein the shell section (111) has a first pore-and-strut structure, PSS, and the inner section (112) has a PSS differing from the first PSS.

### TECHNICAL FIELD

The present invention relates to an implant system for treating bone defects or discontinuities and to a method for producing such an implant system.

### BACKGROUND OF THE INVENTION

Treating bone defects, especially critical size defects, or discontinuities (e.g., after tumor resection) is a key challenge in bone augmentation. Firstly, treatment of bone defects or discontinuities requires stable fixation of implants until the bone defect or discontinuity has been eliminated, for example by biological activity. Secondly, particularly stable implant systems are frequently particularly impermeable and thus prevent natural healing.

DE 10 2013 104 801 A1 discloses a medical mesh-body implant made of a regular three-dimensional mesh structure. However, the single-piece configuration of this implant and its complicated structure, which requires complete active manufacturing, greatly limit both the possible design and the choice of material.

Referring to FIG. 4, EP 3 733 099 A1 discloses an implant system having scaffolding into which individual modules having a porous microstructure can, for example, be clipped in. With this implant system as well, although the uniformity of the modules brings about simplified conditions for production, it also accordingly allows only rather rough matching of the implant to the surrounding bone tissue.

### SUMMARY OF THE INVENTION

It is an object of the present invention to provide an improved implant system for treating bone defects or discontinuities. This object is achieved by an implant system having the features of claim 1. It is a further object to provide a method for producing such an implant system. This object is achieved by a method having the features of the other independent claim.

Accordingly, there is provided an implant system for treating bone defects or discontinuities, comprising a first implant element which is insertable or inserted into a bone defect or a discontinuity of a human bone and a second implant system which is fixable or fixed to the human bone.

The first implant element is attachable or attached to the second implant element by means of at least one connection means, in particular biodegradable connection means, in order to fix the first implant element relative to the human bone (1).

The first implant element comprises a shell section and at least one inner section at least partially enclosed by the shell section.

The shell section has a first pore-and-strut structure, PSS, and the inner section has a second pore-and-strut structure, PSS, differing from the first pore-and-strut structure.

The shell section can completely enclose the inner section, that is to say on all sides, or only partially enclose the inner section, for example in such a way that the inner section is mounted on the edge of the shell section and is therefore for example enclosed by the shell section in five of six spatial directions and is adjacent to another structure in the sixth spatial direction, for example adjacent to the second implant element and/or a bone section (or multiple bone sections).

In connection with the present description, what is to be meant by biodegradable is that the relevant biodegradable element, in this case at least the connection means for attaching the first implant element to the second implant element, is not only tolerated very well by the body into which it is implanted, but is also gradually degraded and/or resorbed thereby. This has in particular the advantage that the relevant biodegradable elements do not necessarily have to be taken out again at a later time, but can for example have been replaced by regrown endogenous tissue. The terms “bioresorbable” and “biodegradable” are both used hereinafter. “Bioresorbable” essentially means physiological uptake of the degradation products by cells, and “biodegradable” refers to mainly extracellular degradation without any physiological incorporation of the degradation products. In the context of the present invention, the two terms are at least interchangeable in that an element described as “bioresorbable” can also (alternatively or additionally) be “biodegradable”, and vice versa.

The implant system is preferably intended for use on mammals, especially on human patients. Whenever mention is made here and hereinafter of “patients” or “a patient”, it will be appreciated that this is intended to cover both male and female patients.

Multiple biodegradable connection means can also be provided between the first implant element and the second implant element, and the biodegradable connection means can have different biodegradation properties, in particular can be biodegradable at different rates. Here, a distinction can for example be made between, firstly, connection means which connect the second implant element to the shell section and, secondly, connection means which connect the second implant element to a respective inner section. Alternatively or additionally, a distinction can also be made between, firstly, connection means which are arranged comparatively closer to a contact site with the human bones (preferably higher biodegradability) and, secondly, connection means which are arranged comparatively further away therefrom (preferably lower biodegradability).

Pore-and-strut structure, PSS, is to be understood to mean a structure according to which there is regular alternation—at least sectionally or everywhere—between voids (“pores” or “pore structures”) and void-enclosing links and braces in between (“struts”). Especially when the PSS is additively manufactured, it is thus possible to precisely define and configure defined pores or voids, struts and the like, with local differences, especially also gradients, being realizable with regard to density (e.g., proportion by volume of struts per unit volume), to material nature and also to the type of regularity of the PSS. The implant system can thus be optimally matched to the circumstances at the site to be treated (bone defect or discontinuity).

Accordingly, the invention also provides, according to a second aspect, a method for producing an implant system according to the invention. Said method comprises at least active manufacturing of at least a portion of the first implant element, especially the shell section and/or the inner section (or one of multiple inner sections). The method can also comprise additive or subtractive manufacturing of the second implant element. Individual subcomponents of the first implant element, especially the shell section and the inner section, can be constructed using different methods of additive manufacturing. Such subcomponents constructed using different methods of additive manufacturing can subsequently be connected to one another with structure-specific biodegradability in a further work step (partial intrinsic fixation).

Advantageous and preferred embodiments, variants or developments of embodiments are more particularly elucidated in the dependent claims and in the description that follows, especially with reference to the figures.

According to some preferred embodiments, variants or developments of embodiments, one of the first and second pore-and-strut structures, PSS, has pore structures of greater than or equal to 200 μm in diameter. The diameter of the pore structure can, in particular, be 300 μm, 400 μm, 500 μm, 600 μm, 700 μm, 800 μm, 900 μm, 1000 μm, 1250 μm, 1500 μm, 1750 μm or 1200 μm. In this connection, the corresponding pore-and-strut structure can also be referred to as “macrostructure”.

Optionally, the other of the first and second pore-and-strut structures has pore structures of less than or equal to 150 μm in diameter. The other PSS can, in particular, have pore structures of less than or equal to (approximately or exactly) 100 μm in diameter or less. The diameter of the pore structure can, for example, be 20 μm, 30 μm, 40 μm, 50 μm, 60 μm, 70 μm, 80 μm, 90 μm or 100 μm. In this connection, the corresponding pore-and-strut structure can also be referred to as “microstructure”. Such a microstructure can advantageously have or undertake depot functionality for bioactive substances. The localized administration of medicaments is possible too, for example medicaments such as antibiotics embedded in the microstructure or introduced therein by dipping.

These differences in the diameters of the pore structures in the different PSSs mean that it is possible to influence in a defined manner which types of tissue can penetrate and migrate into the shell section or the inner section and how rapidly. Since the PSSs were each preferably generated in a defined manner, especially by additive manufacturing, what can be defined as the pore size of the respective pore structures is, for example, the diameter that can be assumed by the largest possible imaginary sphere that could cross the PSS. The pores can, for example, be square (in this case, the pore size would be equal to the edge length of the square), rectangular, round, oval or the like. In the case of a circular cross section, the pore size would accordingly be essentially the diameter of the circle, and in the case of an elliptic cross section, the diameter along the shorter semi-minor axis.

The strut width (or: strut structure diameter) is modulatable too; in particular, a specific ratio of pore size to strut width can be set. In the case of struts having a square cross section, the strut width is equal to the edge length of the square, and in the case of struts having a rectangular cross section, the strut width can be defined as the shorter edge length of the rectangle. Preferred strut widths are greater than 100 μm, but especially 200-300 μm, 400 μm, 450 μm and 500 μm. It is also advantageous when the strut width at least three times the diameter of cells which are to migrate into the corresponding pore-and-strut structure, PSS, especially when the struts have a rounded surface. This allows good cell adhesion.

Specific setting of the ratios between pore size and strut width means that cell migration kinetics (e.g., speed of migration of cells) and degradation rate can be set. Ratios of pore size to strut width that are particularly preferred for the quickened migration of cells are between 1:1 and 6:1; particular preference is given herein to 1:1, 2:1, 4:1, 6:1 and 6:1, with even greater preference being given to ratios between 2.5:1 and 4.5:1. Ratios of pore size to strut width that are preferred for slowed cellular migration are, for example, between 1:2 and 1:4; among these ratios, 1:2, 1:3 and 1:4 are especially advantageous. The ratio of pore size to strut width of the shell section can be set for quickened migration and the ratio of pore size to strut width of the inner element can be set for slowed migration, or vice versa.

In the foregoing and in the following, terms are sometimes abbreviated with acronyms, for instance “PSS” for “pore-and-strut structure”. The long form is usually used, followed by the associated acronym. However, in most cases, only the acronym will be used to improve legibility, whereas in other cases, the acronym is dispensed with. In any case, acronym and long form are intended to be synonymous.

According to some preferred embodiments, variants or developments of embodiments, the first and/or the second pore-and-strut structure, PSS, have a gradient in the diameter of their pore structures. It will be appreciated that when designing a gradient in the PSS, with for example the pore structures changing spatially, in particular increasing or decreasing in size, it is also possible to locally define such a diameter of the pore structures for each subregion.

The total amount of voids, which is formed by the pore structures in the pore-and-strut structure, PSS, can also be referred to as absorption volume capacity and defines, for example, how well blood, water and/or cell tissue can diffuse through the particular PSS. The volume ratio between pore(s) and strut in each unit volume of a PSS can, for example, be between 1:40 and 40:1, in particular between 1:10 and 10:1, further preferably between 1:5 and 5:1, for example 1:1. The ratio also defines, inter alia, the capillary forces and cohesion forces that act in the particular PSS. This can, for example, compensate for a hydrophobic surface effect of polymers. In the case of so-called dip-coating methods, implants are dipped into a functional liquid before implantation, for example into an endogenous liquid, into a medicament such as an antibiotic and/or the like. Since there is usually little time during an operation, it is preferred that the implant has been completely wetted with the functional liquid in a particularly rapid manner in the dip-coating method. To this end, the mentioned ratios and pore-and-strut structures in the mentioned variants have been found to be convenient.

The shell section can be provided with a coating on one or more outer faces. The coating can comprise or consist of:
calcium sulfate;
calcium silicate;
magnesium sulfate;
magnesium silicate; and/or
magnesium phosphate.

Such a coating can, for example, exhibit an antibacterial effect.

According to some preferred embodiments, variants or developments of embodiments, the first implant element comprises or consists of at least one biocomposite material. The first implant element can also consist of two or more different materials which are site-specific.

According to some preferred embodiments, variants or developments of embodiments, the first and/or the second pore-and-strut structure have a gradient in a distribution of at least one biocomposite material. For example, the first or the second pore-and-strut structure, PSS, can consist of a biocomposite material comprising two material components, the ratio thereof to one another running inhomogeneously within the PSS, that is to say changing in a steady or stepped manner from at least one site to at least one other site. In an extreme case, the PSS solely consists of one of the materials at the first site and solely consists of the other material at the other site, there occurring in between a steady transition or a transition that runs discretely in multiple steps. During production, this can, for example, be achieved by carrying out additive manufacturing using two materials and varying the contribution of each material in each volume unit cell according to the desired gradient.

According to some preferred embodiments, variants or developments of embodiments, the second implant element is nonbiodegradable. The second implant element, which is fixable in the human bone or is fixed therein after insertion of the implant system into the patient, can therefore be particularly stable and robust and therefore provide a defined support for the first implant element. Preferably, the second implant element is designed and attached to the first implant element in such a way that the second implant element does not engage or protrude into the bone defect or the discontinuity, but stays outside. In this way, the second implant element can be surgically removed, for example after complete healing, without endangering or counteracting the treatment of the bone defect or the discontinuity as a result. Another contributory factor here is the fact that the at least one connection means between the second implant element and the first implant element is biodegradable, so that there is preferably no longer a rigid mechanical connection between first and second implant element when the second implant element is removed.

According to some preferred embodiments, variants or developments of embodiments, the second implant element is in the form of a narrow reconstruction plate. Such plates have been found to be advantageous in providing a balance between, firstly, desired strength and robustness and, secondly, a volume that is as small as possible.

The second implant element (e.g., in the form of a reconstruction plate) can preferably comprise or consist of titanium, medical-grade stainless steel, a magnesium alloy and/or a cobalt-chromium alloy (Co—Cr) and can therefore also be fully biodegradable or semibiodegradable. For example, the reconstruction plate can be a 2-hole plate or a 4-hole plate.

Depending on the intended implantation site, the second implant element, for example the reconstruction plate, can also consist of polyetheretherketone (PEEK), especially if the surroundings of the site to be treated (bone defect or discontinuity) can be immobilized for a sufficient period of time. This is, for example, the case for finger bones or arm bones, especially long bones. In the case of sites for which this is not possible, a more rigid second implant element, for example a titanium reconstruction plate, is accordingly more advisable. According to some preferred embodiments, variants or developments of embodiments, the inner section comprises at least one ceramic material and/or one biocomposite material, preferably both. The ceramic material and the biocomposite material can comprise or consist of one of the materials described in the foregoing or in the following.

A biocomposite material can, in particular, comprise (or consist of) a polymer component such as:
a polyester-based polymer component, for example PDLLA, PCL, PLGA, PLLA
a polyurethane,
a polyethylene glycol (PEG) and/or
a polyvinyl acetate, PVA

in combination with an inorganic particulate component such as:
a calcium carbonate, CaCO₃,
a strontium carbonate, SrCO₃,
a magnesium oxide, MgO₂,
a calcium phosphate CaPO₄
a material comprising a Ca²⁺ ion, in particular calcium sulfate or octacalcium phosphate,
a magnesium sulfate,
a phosphate,
a trichloropropane, TCP,
a hyaluronic acid, HA,
an iron oxide, FeOₓ or
a sodium silicide (NaSi)-based biodegradable component.

A preferred biocomposite material can, for example, also be composed of a mixture of two, three or four inorganic ions with a polymer matrix.

In many cases, outer sections of the first implant element tend to have larger diameters for pore structures than inner sections. In particular, in some embodiments, developments or variants of embodiments, the shell section will have pore structures of larger diameters than the inner section. Accordingly, the shell section can, for example, have one of the abovementioned macropore structures and/or the inner section one of the mentioned micropore structures.

According to some preferred embodiments, variants or developments of embodiments, the shell section comprises at least one polymer material and/or one ceramic material.

Polymer materials, in particular polyester-based polymer components, can, for example, comprise or consist of:
poly(lactide-co-glycolide), PLGA,
poly-D-L-lactide, PDLLA,
polyglycolic acid, PGA,
poly-L-lactide, PLLA, and/or
polycaprolactone, PCL.

A substructure of the shell section that comprises a polymer material and a substructure of the shell section that comprises a ceramic material can, for example, be produced as separate substructures which are subsequently welded together in the production method, for example by means of ultrasound. Substructures of the shell section together with substructures of one or more inner sections can also be welded or have been welded with one another by ultrasound. One or more substructures of the shell section that comprise a ceramic material are, in particular, arranged on the outer edge of the shell section, particularly preferably in such a way that, when implanted, they are arranged adjacent to at least a bone contact site and/or to the second implant element.

In some preferred embodiments, it is possible that a proportion of ceramic in the shell section decreases from the outside to the inside, with for example the outermost layer of the shell section consisting solely of ceramic and inwardly receiving an increasing proportion of at least one polymer according to a defined gradient. An advantage of ultrasonic welding of ceramic and polymer substructures is that the ultrasonic welding can be completely resorbable, too.

Further preferred embodiments, variants or developments of embodiments will become apparent from the dependent claims and from the description with reference to the figures.

### BRIEF DESCRIPTION OF THE FIGURES

The invention will be more particularly elucidated below on the basis of exemplary embodiments in the figures of the drawings. Shown here by the partially schematized illustration are:

FIG. 1 a schematic three-dimensional view of an implant system according to one embodiment of the present invention;

FIG. 2 a schematic cross-sectional view through the implant system according to FIG. 1;

FIG. 3 a schematic three-dimensional view of an implant system according to a further embodiment of the present invention; and

FIG. 4 a schematic three-dimensional view of an implant system according to yet a further embodiment of the present invention.

In all the figures, identical or functionally identical elements and devices have been provided with the same reference signs, unless otherwise stated.

### DETAILED DESCRIPTION OF THE FIGURES

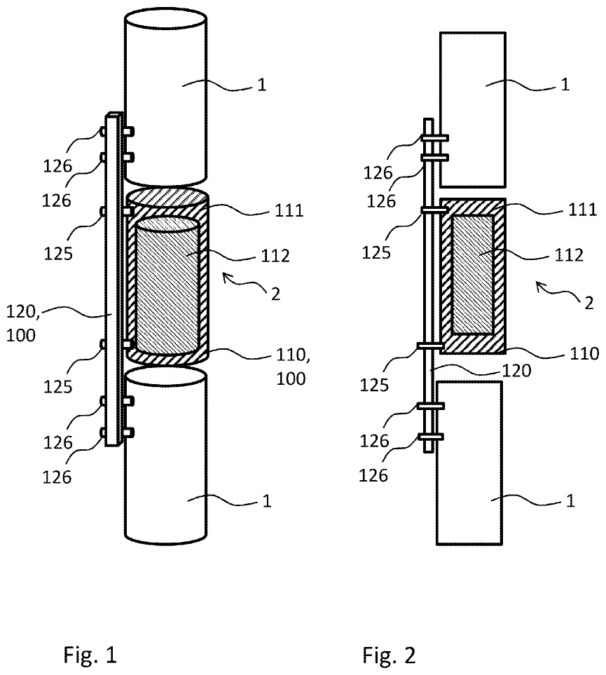

FIG. 1 shows a schematic three-dimensional view of an implant system 100 according to one embodiment of the present invention. FIG. 1 depicts the situation of treating a discontinuity 2 as a result of insertion of the implant system 100 into a patient between two separate sections 1 of a long bone.

FIG. 2 shows the same situation in a schematic cross-sectional illustration.

The implant system 100 comprises a first implant element 110 which has been inserted into the discontinuity 2 after implantation. The first implant element 110 comprises a shell section 111 and an inner section 112 completely enclosed by the shell section 111. Alternatively, it is conceivable that the inner section 112 is directly adjacent to one or both of the interfaces to bone sections 1. In this case, the shell section 111 can also merely partially enclose the inner section 112, thus as a concentric cylindrical shell volume in the example depicted in FIG. 1.

The shell section 111 has a first pore-and-strut structure, PSS, and the inner section 112 has a second pore-and-strut structure differing from the first pore-and-strut structure. Possible here is a multiplicity of differences described in detail in the foregoing and in the following. For example, the first PSS and the second PSS can differ in their pore sizes, so that for instance the first PSS has a microstructure and the second PSS has a macrostructure, or vice versa.

The implant system 100 also comprises a second implant element 120 which is fixable to the human bone, in this case to both bone sections 1, and also fixed thereto in the situated depicted. The second implant element 120 can, for example, be in the form of a planar reconstruction plate and, as has already been described in detail in the foregoing, can, for example, consist of a nonresorbable material. Suitable therefor are, for example, titanium, chromium alloys, magnesium alloys, medical-grade steel and/or the like. When treating a discontinuity 2 which is comparatively easy to immobilize, such as a discontinuity 2 of a finger long bone, it may also be appropriate for the second implant element 120 to be composed of polyetheretherketone, PEEK, for example.

Fixation of the second implant element 120 to the bone, in particular to all separate bone sections 1 of the bone at respectively at least one site, is achieved by connection means 126. Said connection means 126 can, for example, be nonbiodegradable, so that the stability of the fixation of the second implant element 120 to the bone 1 is always maintained. However, the connection means 126 can also be biodegradable.

In contrast, the first implant element 110 is attachable to the second implant element 120, and also attached thereto in the situation in FIG. 1, by means of at least one biodegradable connection means 125. The first implant element 110 can thus be fixed relative to the human bone 1 without the need to establish direct fixation between the first implant element 110 and the human bone itself via a connection means. What is made possible by this is that, when selecting, designing and producing the first implant element 110, no account has to be taken of it being possible in some way for the regions of the first implant element 110 that are adjacent to the human bone 1 to accommodate connection means and to stably anchor them for a sufficient period of time.

Quite the contrary, the pore-and-strut structure, PSS, of the shell section 111 and that of the inner section 112 can therefore be fully directed at the optimal treatment of the discontinuity 2. As can be seen in FIG. 1 and FIG. 2, it is possible that the one or more connection means 125 between first implant element 110 and second implant element 120 mechanically connect and fix merely the second implant element 120 to the shell section 111 in a direct manner, but not the second implant element 120 to the inner section 112 in a direct manner.

Therefore, when designing the first pore-and-strut structure, PSS, of the shell section 111, account can still be taken of the necessary fixation to the second implant element 120, whereas when designing the second pore-and-strut structure, PSS, of the inner section 112, only the concerns of treating the discontinuity 2 can play a role. Fixation of the inner section 112 relative to the rest of the implant system 100 and relative to the human bone can therefore be achieved especially by a form fit, for example by the shell section 111 completely or at least partially enclosing the inner section 112, it also being possible for fixation of the inner section 112 to be ensured at least in part by the human bone itself, for instance by two-sided adjacency to opposing bone sections 1.

The distances between the implant elements 110, 120 from one another and from the human bone 1 are depicted in FIG. 1 and FIG. 2 in a highly exaggerated manner in order to be able to distinguish the individual parts from one another; in reality, the gaps, if present at all, are as small as possible. Preferred values and properties for the first pore-and-strut structure, PSS, of the shell section 111 were explained in detail in the foregoing.

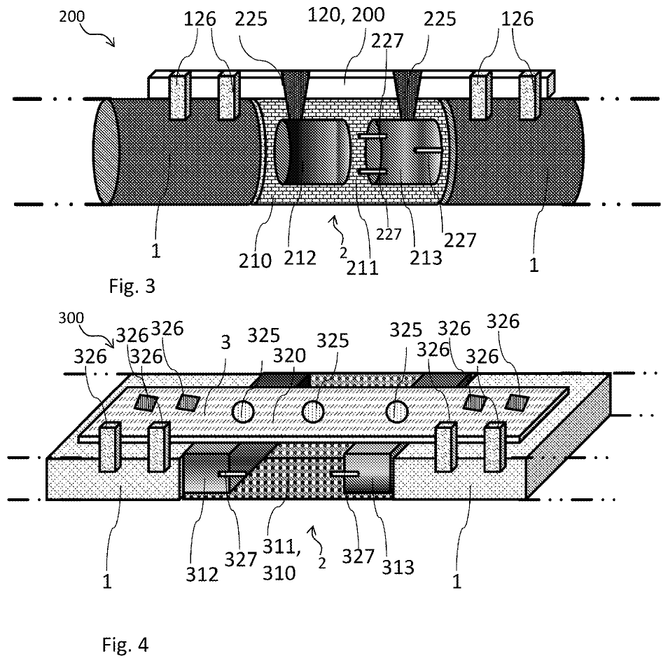

FIG. 3 shows an implant system 200 according to a further embodiment of the present invention, again in a schematical three-dimensional view. The human bone 1 schematically depicted in FIG. 3 is also a long bone, for example of limbs or of ribs.

In contrast to the implant system 100, the first implant element 210 of the implant system 200 comprises two inner sections 212, 213 which are separate from one another and which are both completely enclosed by the shell section 211 of the first implant element 210. In the example depicted in FIG. 3, the two inner sections 212, 213 are substantially or exactly arranged flush with one another in a longitudinal direction, in which the long bone 1 also extends with the discontinuity 2 to be treated. In this example, the two inner sections 212, 213 are of the same size, but it will be appreciated that this does not need to be the case, that more than two inner sections 212, 213 can also be provided, that they do not have to be flush with one another in the longitudinal direction and/or and so forth.

As also in FIG. 1 and FIG. 2, the second implant element 120 of the implant system 200 is connected or attached to the first implant element 210 by means of two biodegradable connection means 225. Each of the biodegradable connection means 225 is adjacent to respectively one inner section 212, 213 of the first implant element 210 of the implant system 200 and can optionally also penetrate to some extent into the respective inner section 212, 213.

As also indicated in FIG. 3 by shading, the inner sections 212, 213 can each have a gradient in their respective pore-and-strut structure, PSS. The first inner section 212 indicates that a gradient (e.g., in pore size and/or in the ratio of the composition of at least two materials, in their density, etc.) extends in the longitudinal direction from one end of the first inner section 212 to the other end of the first inner section 212. By contrast, the second inner section 213 indicates that there is a gradient (again with regard to pore size, the ratio of material composition and/or the like) from the two outermost ends, as seen in the longitudinal direction, toward the middle of the second inner section 213. In the middle—again as defined in the longitudinal direction—of the second inner section 213, there can therefore be, for example, a material composition having increased strength (compared to the end and closing sections of the second inner section 213 in the longitudinal direction). As likewise indicated in FIG. 3, a biodegradable connections means 225 can interconnect not only the second implant element 120 and the shell section 211 but also precisely the middle of the second inner section 213. Whenever a gradient in pore size or strut width is provided, whichever is the other variable can accordingly also be provided with an opposite gradient, for example in such a way that a unit cell width of a cell composed of pore+surrounding strut structures remains the same size (with changing mass density).

In the case too of the first inner section 212, the mentioned gradient can at least also be a gradient in the strength, especially breaking strength, of the second inner section 213. In FIG. 3, it is again schematically depicted that the connection means 225 touches an outer end of the first inner section 212, which outer end is seen in the longitudinal direction and has said increased strength. Therefore, even within the inner section, functionally can additionally be specifically and locally adjusted via the inhomogeneous pore-and-strut structure, PSS.

In FIG. 3, it is also depicted that the second inner section 213 is fixed to the shell section 211 via—for example—three connection means 227 in the form of biodegradable pins.

Here, the pins can, for example, be introduced by ultrasound or be introduced in the form of screws. Specifically, the pins can have a diameter of 1, 1.5, 2 or 2.5 mm with a length of 0.9, 1.2, 1.4, 1.8 or 2.1 mm. Specific utilization of screw fixing in the longer length of the pins is also used, smaller elements of a length equal to or smaller than 1.5 mm are introduced by ultrasound. Preferred values and properties for the various pore-and-strut structures, PSS, of the shell section 211 and for the two inner sections 212, 213 of the inner section were explained in detail in the foregoing.

FIG. 4 shows a schematic three-dimensional illustration of an implant system 300 according to a further embodiment of the present invention.

The implant system 300 shown in FIG. 4 was, by way of example, used for treating a discontinuity 2 in a flat bone, for example a lower jaw or a cranial bone. In contrast to the implant systems 100; 200, in which the first implant element 110; 210 was roughly cylindrical, the first implant element 310 of the implant system 300 is roughly cuboid. The first implant element 310 also comprises two inner sections 312, 313 which are separate from one another and which are each arranged on an outer border of the inner section 311.

In the case of the embodiment shown in FIG. 4, the two inner sections 312, 313 are even each arranged at a different corner edge of the cuboid first implant element 310 and are each also cuboid themselves. Therefore, each of the two interfaces of the first implant element 310 with the bone 1 is formed partly by the shell section 311 and partly by an interface of a respective inner section 312, 313. Furthermore, in the case of the embodiment according to FIG. 4, it is also possible that each of the inner sections 312, 313 is directly adjacent to the second implant element 320. Accordingly, it is possible, for example, for each of the two inner sections 312, 313 to be directly connected to the second implant element 320 via a biodegradable connection means 325, which fixes them to one another, without the shell section 311 being passed through.

A further biodegradable connection means 325 can, in turn, fix the second implant element 320 to a portion of the inner section 311 that is arranged between the two inner sections 312, 313, without the connection means 325 touching any of the inner sections 312, 313. Therefore, each connection means 325 can be specifically chosen with regard to its material properties, etc., in such a way that it is appropriate for whatever is the connection situation. In particular, biodegradability over time can be specifically set. For example, it is possible that the middle connection means 325, which directly connects the second implant element 320 to the inner section 311, has lower biodegradability, that is to say it degrades more slowly, than the other two connection means 325 between the second implant element 320 and one of the two inner sections 312, 313.

In the case too of the embodiment of the implant system 300 shown in FIG. 4, it is schematically depicted that the inner sections 312, 313 each have a gradient, for example with regard to diameter of the pore structures, with regard to ratios of material compositions, with regard to density and/or with regard to further properties of the particular pore-and-strut structure, PSS. As indicated in FIG. 4, a gradient can, for example, extend from a side near the second implant element 320 toward a side of the respective inner section 312, 313 that is facing away from the second implant element 320. Alternatively or additionally, gradients along an extent of a longitudinal direction of the second implant element 320 are also conceivable, however. FIG. 4 also depicts, by way of example, further connection means 327, for example in the form of biodegradable pins, which can bring about fixation of each of the inner sections 312, 313 to the shell section 311. Therefore, the inner sections 312, 313 are not only mechanically fixed by means of the second implant element 320 or by means of the form fit of bone 1, shell section 311 and second implant element 320, but also additionally force-fittingly fixed to the shell section 311 in a direct manner.

The variants depicted in FIG. 1 to FIG. 3 for the respective second implant element 120 can, for example, be in the form of a 2-hole reconstruction plate. FIG. 4 shows an example in which the second implant element 320 is in the form of a 4-hole reconstruction plate. The second implant element 320 can also be biodegradable or nonbiodegradable, suitable materials in the latter case being especially titanium, medical-grade steel, magnesium alloys and/or chromium alloys and/or the like, though polyetheretherketone, PEEK, can also be used.

In the case of the implant system 300, what is inserted through each hole of the 4-hole reconstruction plate of the second implant element 320 is, by way of example, a cuboid connection means 326, for example a nondegradable one, in order to fixedly connect the second implant element 320 to the two sections of the human bone 1. This ensures a particularly good support of the entire implant system 300 on the bone 1, and the second implant element 320 can then, for example, be taken out again from the bone 1 when the connection means 325 have fully biodegraded and the support by the second implant element 320 for the first implant element 310 is no longer needed. Alternatively, the connection means 326 between the human bone 1 and the second implant element 320 can likewise be biodegradable. In this case, it is advantageous when the biodegradability of the connection means 325 between the second implant element 320 and the first implant element 310 is higher, that is to say that biodegradation occurs more rapidly, than biodegradability of the connection means 326 between the second implant element 320 and the human bone 1. This can ensure that the second implant element 320 does not detach from the bone 1 before it has detached from the first implant element 310, and so there is no need to fear that movement of the second implant element 320 will endanger the treatment of the discontinuity by the first implant element 310.

The first pore-and-strut structure, PSS, of the shell section 311 can, for example, have the following properties: pore size (e.g., pore diameter, pore side length) of 500 μm, strut structure diameter of 200 μm or greater, PDLLA-Ca—Mg biocomposite material composed of a mixture of calcium phosphate and magnesium phosphate having a mixture of A:B:C percent by mass, having a calcium phosphate gradient in percentage by mass, for example A=80, B=10, C=10 on the outer face (adjacent to the second implant element 320) of the shell section 311 and A=80, B=5, C=15 on the inner face (side facing away from the second implant element 320) of the shell section 311. Magnesium sulfate can preferably be applied as a coating to the inner face of the shell section 311, for example in order to achieve anitbacterial effects.

The second pore-and-strut structure of the first inner section 312 can, for example, have the following properties: pore size of 400 μm, strut structure diameter of 150 μm, and PCL material. The second pore-and-strut structure of the second inner section 313 (also referable to as third pore-and-strut structure) can, for example, have the following properties: maximum pore size of 300 μm, minimum strut structure diameter of 100 μm, PDLLA-CaCO₃ biocomposite materials having a mixture of 72:18 percent by mass, gradient in the sense of a gradually (or stepwise) increasing strut structure diameter from 100 μm up to 300 μm from the outside (adjacent to the second implant element 320) to the inside with pore size concurrently becoming smaller in a converse manner.

The respective first implant element 110; 210; 310 containing the inner section 111; 211; 311 and the inner section(s) 112; 212; 213; 312; 313 can be produced by means of various additive manufacturing techniques.

1. An implant system for treating bone defects or discontinuities, comprising:
a first implant element configured for insertion into a bone defect or a discontinuity of a human bone,
a second implant element configured to be fixed to the human bone,
wherein the first implant element is attachable to the second implant element by means of at least one biodegradable connection means in order to fix the first implant element relative to the human bone,
wherein the first implant element comprises a shell section and an inner section at least partially enclosed by the shell section,
wherein the shell section has a first pore-and-strut structure, PSS, and the inner section has a second pore-and-strut structure, PSS, differing from the first pore-and-strut structure.

2. The implant system according to claim 1, wherein one of the first and second pore-and-strut structures has pore structures of greater than or equal to 200-700 micrometers in diameter.

3. The implant system according to claim 1, wherein one or both of the first and the second pore-and-strut structure has a gradient in the diameter of the pore structures.

4. The implant system according to claim 1, wherein the first implant element includes at least one biocomposite material.

5. The implant system according to claim 4, wherein one or both of the first and the second pore-and-strut structure has a gradient in a distribution of at least one biocomposite material.

6. The implant system according to claim 1, wherein the second implant element is nonbiodegradable.

7. The implant system according to claim 1, wherein the second implant element is in the form of a narrow reconstruction plate.

8. The implant system according to claim 1, wherein the shell section includes at least one polymer material.

9. The implant system according to claim 1, wherein the inner section includes at least one ceramic material.

10. A method for producing an implant system according to claim 1, comprising additive manufacturing of at least a portion of the first implant element.

11. The implant according to claim 2, wherein the other of the first and second pore-and-strut structures has pore structures of less than or equal to 150 micrometers in diameter.

12. The implant system according to claim 1, wherein the shell section includes at least one ceramic material.

13. The implant system according to claim 1, wherein the inner section includes at least one biocomposite material.
